(19) United States
(12) Patent Application Publication
(10) Pub. No.: US 2022/0062067 A1
(43) Pub. Date: Mar. 3, 2022

(54) URINARY INCONTINENCE DEVICE

(71) Applicant: Lyle K. Scholl, Noblesville, IN (US)

(72) Inventor: Lyle K. Scholl, Noblesville, IN (US)

(73) Assignee: Lyle K. Scholl, Noblesville, IN (US)

(21) Appl. No.: 17/445,335

(22) Filed: Aug. 18, 2021

Related U.S. Application Data
(60) Provisional application No. 63/069,779, filed on Aug. 25, 2020.

(51) Int. Cl. A61F 13/471; A61F 13/514; A61F 13/64

(52) U.S. Cl. CPC A61F 13/471; A61F 2013/15121; A61F 13/64; A61F 13/51456

(57) ABSTRACT

In one aspect, the present disclosure provides a device for collection of urine from a male user. In accordance with some forms of the disclosure, the device is configured for temporary attachment to the penis of a user upon completion of urination to capture any additional urine output that may persist after urination.

### BACKGROUND

Urinary incontinence is a significant problem that has the potential to be very embarrassing for the individual suffering from the condition. Those who suffer from urinary incontinence run the spectrum from highly active to incapacitated. Regardless, those enduring urinary incontinence require solutions that allow them to live their lives with minimal disruption from this condition.

Post Micturition Dribble (PMD) is a common condition experienced by healthy men, and is characterized by the involuntary loss of urine immediately after urination, usually after leaving the toilet. PMD is likely to occur when the penis is being replaced and clothes rearranged after visiting the toilet. PMD may be due to a weakness of the muscles of the pelvic floor, which surround the urethra. The pelvic floor muscles can be weakened by: operations for an enlarged prostate, continual straining to empty the bowels especially when constipated, constant cough, for example, a smoker's cough, overweight, neurological damage, and/or persistent heavy lifting. Other signs of urinary dysfunction are not normally present, although some men have symptoms such as urgency, frequency, hesitancy and straining. For older men, they may develop PMD following a prostatectomy and may have associated urinary symptoms.

The myriad of absorbent devices designed to assist those with urinary incontinence suffer from any number of deficiencies, including being too bulky for active individuals and being indiscrete. The issue of discretion is particularly acute for males, especially when the need to change the absorbent device arises in public. Thus, there is a need for an improved disposable absorbent device for ambulatory males.

### SUMMARY

In one aspect, the present disclosure provides a device for collection of urine from a male user. In accordance with some forms of the disclosure, the device is configured for temporary attachment to the penis of a user upon completion of urination to capture any additional urine output that may persist after urination. Accordingly, in one embodiment the present disclosure provides a urinary incontinence device comprising a body member extending from an open end to a closed end, the open end configured for receiving the glans penis of a user, the device configured for attachment to the penis of the user. In certain embodiments, the device comprises a drawstring positioned near the open end and configured to secure the body member over the glans penis of the user. In some forms, the drawstring may comprise an elastic material. In accordance with certain embodiments, the device includes a retention member configured to receive the drawstring, and allow the drawstring to pass through in one direction while resisting movement of the drawstring material in the opposite direction. In some forms, the retention member comprises a release mechanism configured to release the drawstring from the retention member upon activation. In accordance with certain embodiments, the device may comprise a multilayer sheet form material, having an inner surface configured to absorb fluid and a fluid impermeable outer surface.

In certain embodiments, the urinary incontinence device comprises at least one flap extending from the body member and configured to extend around the penis of the user to secure the body member over the glans penis of the user. In some forms, the device comprises a first flap and a second flap. In accordance with some forms, the device comprises an adhesive portion on the at least on flap, the adhesive portion comprising an adhesive material configured to contact a corresponding portion of the urinary incontinence device to secure the body member over the glans penis of the user.

In alternative embodiments, the present disclosure provides methods of using the devices disclosed herein. For example, the present disclosure provides, a method of alleviating urinary incontinence, the method comprising securing a urinary incontinence device as described herein to the penis of a user.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a perspective view of one embodiment of a urinary incontinence device as provided by the present disclosure.

FIG. 2 is a top down view of one embodiment of a urinary incontinence device as provided by the present disclosure.

FIG. 3 is a side view of one embodiment of a urinary incontinence device as provided by the present disclosure.

FIG. 4 is a side view of one embodiment of a urinary incontinence device as provided by the present disclosure.

FIG. 5 is a cross-sectional view along line 5 of FIG. 2.

FIG. 6A is a front view of one embodiment of a urinary incontinence device as provided by the present disclosure.

FIG. 6B is a top down view of one embodiment of a urinary incontinence device as provided by the present disclosure.

### DESCRIPTION OF THE SELECTED EMBODIMENTS

For the purpose of promoting an understanding of the principles of the disclosure, reference will now be made to the embodiments illustrated in the figures and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one skilled in the art to which the invention relates. Certain embodiments of the disclosure are shown in detail; although it will be apparent to those skilled in the relevant art that some features that are no relevant to the present disclosure may be shown for the sake of clarity.

As disclosed herein, in certain aspects, the present disclosure provides devices for alleviating mild urinary incontinence, including post micturition dribble (PMD). Such devices are configured for discreate and temporary application by the user. Devices of the present disclosure are intended for use with light incontinence, for example for application upon completion of urination to capture any additional urine leakage that may persist. Because the devices are designed with a low profile, they may be easily stored in a pocket and discretely applied and removed by the user without requiring removal of the pants or underwear of the user. Such leakage may occur with increased frequency for example after prostate or other surgery, or may occur naturally.

As used herein, the term “glans penis” generally refers to the bulbous structure at the distal end of the penis. As used herein the term refers to users who have been circumcised as well as those who have not been circumcised to describe the relative position of the urinary incontinence device. Depending on the anatomy of the user, the device may be placed over foreskin overlying the glans penis.

In certain aspects, the present disclosure provides a urinary incontinence device for a male patient comprising a body member having an absorbent inner surface. In some forms, the body member has an open end and a closed end, the open end being configured for insertion of at least a portion of the penis of the user. In accordance with some forms the device is configured to receive the glans penis of the user, without require further insertion of the penis. In this way, the devices of the present disclosure may be configured to extend only over the glans penis thus providing a low profile discreate solution to light incontinence. In accordance with some forms, the body member may be collapsible, or foldable to a flat configuration. In use, the device would be unfolded or expanded to allow insertion of the penis.

Devices of the present disclosure may be provided in a variety of sizes. In preferred embodiments, the devices provided herein are sized to fit over substantially all of the glans penis of a user. In accordance with some forms, the present disclosure provides a urinary incontinence device having a body member length from an open end to a closed end. In some forms, the body member length of less than about 10 cm, preferably less than about 9 cm, even more preferably less than about 8 cm. In some forms, the body member length is about 3 cm to about 10 cm, preferably about 4 cm to about 9 cm, even more preferably about 5 cm to about 8 cm.

The absorbent inner surface of the body member may comprise any absorbent material capable of absorbing urine. The absorbent inner layer may be formed of mixtures of fiberized pulp and superabsorbent polymers, such as those materials used for conventional disposable diapers and/or feminine hygiene products. Suitable absorbent materials include but are not limited to polyacrylate/polyacrylamide copolymers, ethylene maleic anhydride copolymer, cross-linked carboxymethyl cellulose, polyvinyl alcohol copolymers, cross-linked polyethylene oxide, and/or starch grafted copolymer of polyacrylonitrile.

In some forms, the device may include a fluid impermeable or fluid-resistant outer layer. While not necessary to broader aspects of the disclosure, the body member may include such fluid impermeable outer layer or fluid resistant outer layer. In some forms the body member is formed from one or more multilayer sheet materials, for example a sheet material having at least one absorbent layer and at least one fluid impermeable or fluid resistant layer.

The body member may take any one of a variety of suitable shapes. In accordance with some forms, the body member comprises a conical shape with the open end comprising the wider base portion of the cone. In alternative embodiments, the body member comprises a pocket formed from one or more sheet materials, the pocket having an open end and a closed end. In certain embodiments, the absorbent inner surface comprises the inner wall of body member. In other embodiments, the absorbent material may comprise an absorbent pad contained within the body member.

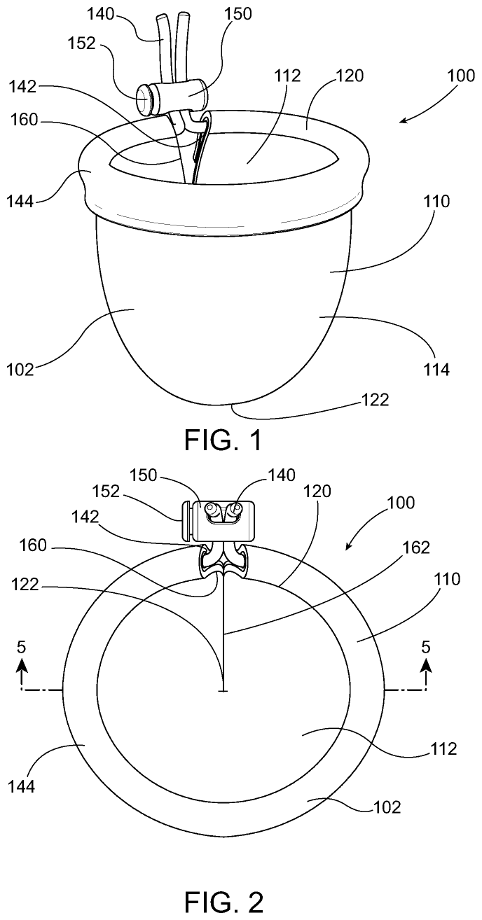

With reference now to the embodiment illustrated in FIG. 1, shown is a urinary incontinence device 100, having a body member 110 extending from an open end 120 to a closed end 122. In the illustrated embodiment, the body member comprises a multilayer material 102 having an inner surface 112, which may comprise an absorptive material as disclose herein, and an outer surface 114, which may comprise a fluid impermeable material as disclosed herein. The illustrated embodiment includes drawstring 140, secured by retention member 150. In the illustrated embodiment, the retention member includes release member 152. In the illustrated embodiment drawstring channel 142 extends around the circumference of the open end, with the drawstring passing through the channel. The channel may be formed by any suitable manner, for example, the multilayer material may include a fold 144 near the open end. The drawstring may be secured in any suitable manner, for example tied off or secured with a retention member as illustrated. The body member of the device may include a slit 160, configured to allow the circumference of the open end to expand and contract, such that then the drawstring is pulled the circumference of the open end shrinks.

As disclosed above, in some forms urinary incontinence devices may include a retention member. In some forms, the retention member is configured to secure the drawstring, or other securing mechanism, in a tight secure configuration on the penis. The retention member may be configured to allow the drawstring to pass through in one direction, for example when securing the device over the penis, while resisting movement of the drawstring material in the opposite direction. In this way, the device is secured in position. In some forms, the retention member comprises a release mechanism, configured to release the drawstring to facilitate removal of the device.

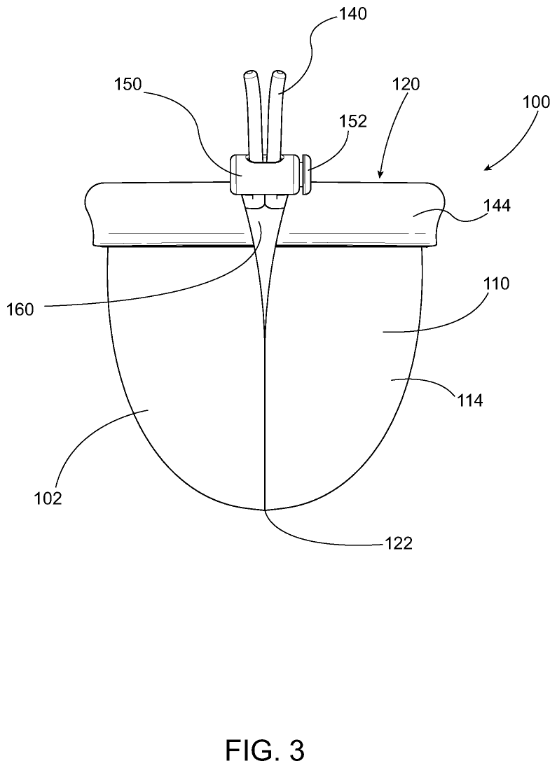

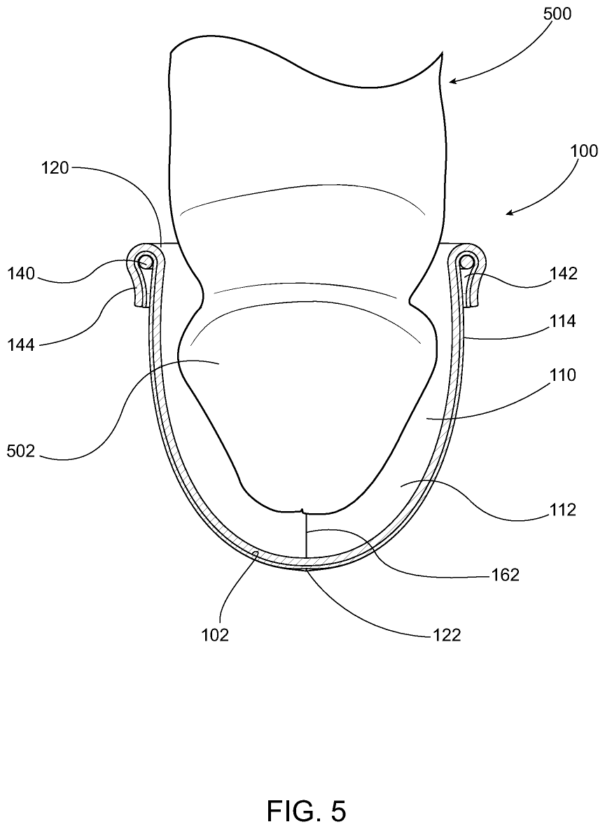

Turning now to a discussion of the embodiments shown in FIGS. 2, 3, 4, and 5, shown is an embodiment of a urinary incontinence device as disclosed herein. FIG. 2 is a top down view. FIG. 3 is a side view. FIG. 5 is a cross section of the device along line 5 of FIG. 2, and includes for reference penis 500, and glans penis 502. In the illustrated embodiments, a urinary incontinence device 100, having a body member 110 extending from an open end 120 to a closed end 122. The body member comprises a multilayer material 102 having an inner surface 112, which may comprise an absorptive material as disclose herein, and an outer surface 114, which may comprise a fluid impermeable material as disclosed herein. The illustrated embodiment includes drawstring 140, secured by retention member 150. In the illustrated embodiment, the retention member includes release member 152. In the illustrated embodiment drawstring channel 142 extends around the circumference of the open end, with the drawstring passing through the channel. The channel may be formed by any suitable manner, for example, the multilayer material may include a fold 144 near the open end. The drawstring may be secured in any suitable manner, for example tied off or secured with a retention member as illustrated. The body member of the device may include a slit 160, configured to allow the circumference of the open end to expand and contract, such that then the drawstring is pulled the circumference of the open end shrinks. In accordance with certain embodiments seam 162 extends from slit 160 to closed end 122.

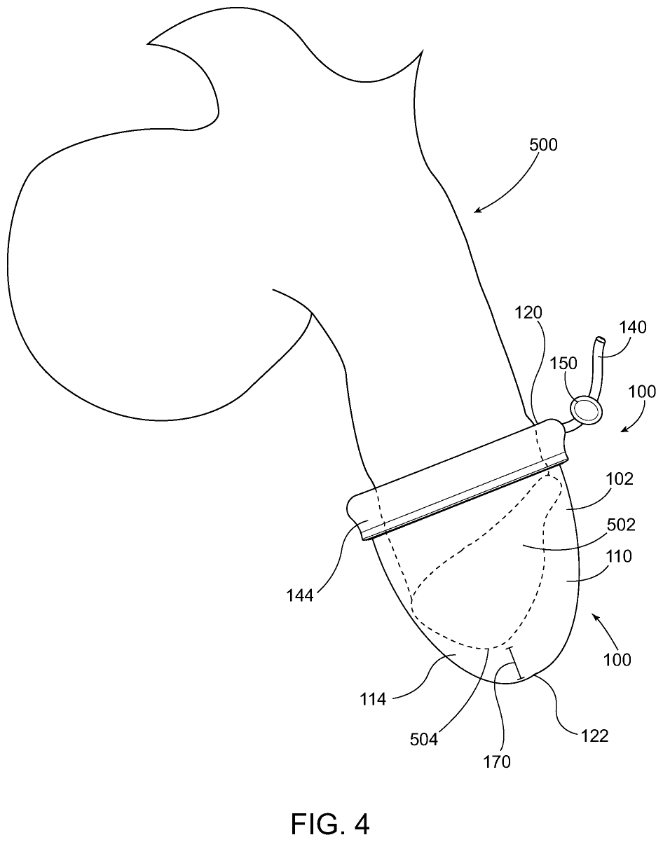

FIG. 4 illustrates one embodiment of a urinary incontinence device as disclosed herein in use on a patient penis 500, having glans penis 502. In use, the device fits over the glans penis area as illustrated. In the illustrated embodiment, the device is configured to allow for gap 170 between the distal tip 504 of the penis and the closed end 122 of the device. Such a configuration prevents fluid accumulation around the urethral opening thus preventing infection and irritation.

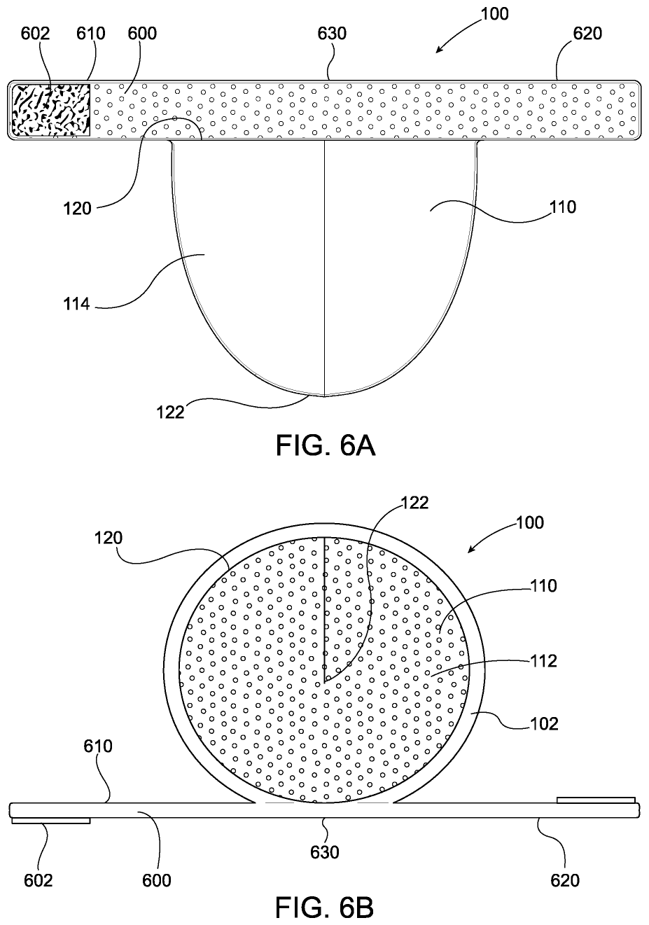

Turning now to a discussion of the embodiments shown in FIGS. 6A and 6B. FIG. 6A is a side view of one embodiment of a urinary incontinence device as described herein. FIG. 6B is a top-down view of the urinary incontinence device of FIG. 6A. Device 100 comprises body member 110 extending from open end 120 to closed end 122. In the illustrated embodiment, the body member comprises a multilayer material 102 having an inner surface 112, which may comprise an absorptive material as disclose herein, and an outer surface 114, which may comprise a fluid impermeable material as disclosed herein. In certain embodiments, a device of the present disclosure may include an attachment member 600, configured to extend around the penis to secure the device in position over the glans penis. In certain embodiments, the attachment member may be comprised of the same multilayer material of which the body member is comprised. The attachment region may comprise a first flap 610 and a second flap 620, extending from a central region 630. Although it is also within the scope of the disclosure to provide a single, longer flap configured to wrap around the penis. In some forms, the attachment member may be comprised of a different material than that of the body member, for example, elastic or more conformable materials may be preferred. In accordance with some forms, one or more adhesive portions 602 may be present on the attachment member. Such adhesive portions may be configured to adhere to the opposing flap. In some forms, the opposing flap contains another adhesive region to facilitate adherence of the device. Such adhesives are preferable configured to provide a secure hold but are easily separable upon removal of the device.

As disclosed herein, the present invention provides urinary incontinence devices configured to attach to the penis of a user. In accordance with some forms, the body member incudes an adhesive surface configured to adhere the device to the penis of the user. In some forms, an adhesive ring is present around the open end of the body member. In some forms, an adhesive strip is disposed within the body member. It is within the scope of the disclosure that the adhesive layer may be covered by a disposable peel strip, or may be contained within the collapsed body member and exposed upon expansion of the device as discussed herein. Suitable adhesives include those suitable for application to the skin without leaving an adhesive residue upon removal, for example acrylate and/or methacrylate. It is within the scope of the disclosure that the adhesive surface comprises an adhesive tape, glue, and/or gel suitable for contact with skin.

In addition, or alternatively to an adhesive, the body member may be configured for attachment to the penis with a compliant attachment member. A compliant attachment member as used herein includes materials that may be deformed to secure the penis within the body member. The compliant member may be positioned at or near the open end of the body member. Exemplary materials for a compliant member include but are not limited to metals, such as aluminum or galvanized iron, and other suitable plastics. A compliant attachment member may be disposed substantially around the entire open end or may include on or more sections of complaint material which may be deformed to secure the penis within the body member.

In some forms, the present disclosure provides a urinary incontinence device comprising an elastic attachment member elastic attachment member may be positioned at or near the open end of the body member. For example, in some forms the present disclosure provides urinary incontinence devices having an elastic attachment member positioned as a ring with in a perimeter edge defining the open end. In accordance with some forms, one or more elastic members are positioned to extend over the open end of the body member. In accordance with such forms, an elastic member may extend from a first attachment point at or near the perimeter edge to a second attachment point at or near the perimeter edge. When present, a second elastic attachment member may extend from a third attachment point to a fourth attachment point over said open end such that the first and second elastic attachment members span the open end. In some forms, the first and second attachment members are generally parallel to one another. In this way, when a penis is inserted the one or more elastic attachment members is deflected and secures the penis within the body member.

In accordance with some forms, the present disclosure provides a urinary incontinence device comprising a plurality of attachment tabs. In some forms, the attachments tabs extend from at or near the perimeter edge of the body member and over the open end. In certain embodiments, the device comprises two or more attachment tabs, preferably three or more. In some forms, each of the tabs comprises a base portion attached to the body member and a free edge opposite the base portion, the free edge extending over the open end of the body member. In this way, upon insertion of a penis the resilient attachment tabs are deflected and secure the body member over the penis. The attachment tabs may be formed from any suitable resilient material.

The present disclosure also provides a method of alleviating light urinary incontinence using any of the devices disclosed herein. Such methods comprise securing the urinary incontinence device to the penis of the user. In preferred embodiments, the urinary incontinence device is secured to the glans penis of the user.

### Embodiments

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein

1. A urinary incontinence device for a male user, the device comprising:

a body member extending from an open end to a closed end and having an absorbent inner surface, said open end configured for receiving the glans penis of the user, and wherein the device is configured for attachment to the penis of the user.

2. The urinary incontinence device of embodiment 1, wherein the body member comprises an adhesive layer configured to adhere the device to the penis of the user.
3. The urinary incontinence device of embodiment 1, wherein the body member comprises a compliant attachment member.
4. The urinary incontinence device of embodiment 3, wherein the compliant attachment member is positioned near said open end.
5. The urinary incontinence device of embodiment 4, wherein said open end is defined by a perimeter edge and wherein said compliant attachment member extends along a portion of said perimeter edge.
6. The urinary incontinence device of embodiment 1, comprising an elastic attachment member.
7. The urinary incontinence device of embodiment 6, wherein said elastic attachment member extends from a first attachment point to a second attachment point over said open end.
8. The urinary incontinence device of embodiment 7, comprising a second elastic attachment member extending from a third attachment point to a fourth attachment point over said open end.
9. The urinary incontinence device of embodiment 8, wherein said first elastic attachment member extends generally parallel to said second elastic attachment member over said open end.
The urinary incontinence device of embodiment 6, wherein said open end is defined by a perimeter edge and wherein said elastic attachment member forms a ring within said perimeter edge.
11. The urinary incontinence device of embodiment 1, comprising a plurality of attachment tabs extending from near said perimeter edge and over said open end.
12. The urinary incontinence device of embodiment 11, wherein said tabs comprise a resilient material.
13. The urinary incontinence device of any one of the preceding embodiments, wherein said body member has a conical configuration extending from said open end to said closed end.
14. The urinary incontinence device of any one of the preceding embodiments, wherein said body member comprises a sheet-form material pocket.
15. The urinary incontinence device of any one of the preceding embodiments wherein the body member is configured to expanded from a collapsed state to an expanded state.
16. The urinary incontinence device of any one of the preceding embodiments wherein the device is configured to fit over the glans penis of the user.
17. The urinary incontinence device of any one of the preceding embodiments, wherein the body member has a body member length from said open end to said closed end, and wherein said body member length is less than 8 cm.
18. The urinary incontinence device of embodiment 1, comprising a drawstring positioned near said open end and configured to secure said body member over the glans penis of the user.
19. The urinary incontinence device of embodiment 18, wherein said drawstring comprises an elastic material.
20. The urinary incontinence device of any one of embodiments 18 or 19, comprising a retention member configured to receive said drawstring, said retention member configured to allow the drawstring to pass through in one direction, and resisting movement of the drawstring material in the opposite direction.
21. The urinary incontinence device of embodiment 20, wherein said retention member comprises a release mechanism configured to release the drawstring from the retention member upon activation.
22. The urinary incontinence device of any one of embodiments 1 through 21, wherein the device comprises a fluid impermeable outer layer.
23. The urinary incontinence device of embodiment 1, wherein the device comprises at least one flap extending from said body member and configured to extend around the penis of the user to secure the body member over the glans penis of the user.
24. The urinary incontinence device of embodiment 23, wherein the device comprises a first flap and a second flap.
25. The urinary incontinence device of any one of embodiments 23 or 24, wherein the device comprises an adhesive portion on said at least on flap, said adhesive portion comprising an adhesive material configured to contact a corresponding portion of the urinary incontinence device to secure the body member over the glans penis of the user.
26. The urinary incontinence device of embodiments 1 through 25, wherein the device is configured to provide a gap between the distal tip of the penis of the user and the closed end of the body member.
27. A method of alleviating urinary incontinence, the method comprising:

securing the urinary incontinence device of any one of the preceding embodiments to the penis of a user.

While the invention has been shown and described in detail in the figures and foregoing description, the same is to be considered as illustrative and not restrictive in character, it is being understood that only the preferred embodiment has been shown and described and that all changes, equivalents, and modifications that come within the spirit of the invention defined by the following claims are desired to be protected.

1. A urinary incontinence device for a male user, the device comprising:
a body member extending from an open end to a closed end and having an absorbent inner surface, said open end configured for receiving the glans penis of the user, and wherein the device is configured for attachment to the penis of the user.

2. The urinary incontinence device of claim 1, comprising a drawstring positioned near said open end and configured to secure said body member over the glans penis of the user.

3. The urinary incontinence device of claim 2, wherein said drawstring comprises an elastic material.

4. The urinary incontinence device of claim 2, comprising a retention member configured to receive said drawstring, said retention member configured to allow the drawstring to pass through in one direction, and resisting movement of the drawstring material in the opposite direction.

5. The urinary incontinence device of claim 4, wherein said retention member comprises a release mechanism configured to release the drawstring from the retention member upon activation.

6. The urinary incontinence device of claim 1, wherein the device comprises a fluid impermeable outer layer.

7. The urinary incontinence device of claim 1, wherein the device comprises at least one flap extending from said body member and configured to extend around the penis of the user to secure the body member over the glans penis of the user.

8. The urinary incontinence device of claim 7, wherein the device comprises a first flap and a second flap.

9. The urinary incontinence device of claim 7, wherein the device comprises an adhesive portion on said at least on flap, said adhesive portion comprising an adhesive material configured to contact a corresponding portion of the urinary incontinence device to secure the body member over the glans penis of the user.

10. The urinary incontinence device of claim 1, wherein the device is configured to provide a gap between the distal tip of the penis of the user and the closed end of the body member.

11. The urinary incontinence device of claim 1, wherein said body member has a conical configuration extending from said open end to said closed end.

12. The urinary incontinence device of claim 1, wherein said body member comprises a sheet-form material pocket.

13. The urinary incontinence device of claim 1 wherein the body member is configured to expanded from a collapsed state to an expanded state.

14. The urinary incontinence device of claim 1 wherein the device is configured to fit over the glans penis of the user.

15. The urinary incontinence device of claim 1, wherein the body member has a body member length from said open end to said closed end, and wherein said body member length is less than 8 cm.

16. A method of alleviating light urinary incontinence, the method comprising:
securing the urinary incontinence device of any one of the claim 1 to the penis of a user.
